(19) United States
(12) Patent Application Publication
(10) Pub. No.: US 2015/0057759 A1
(43) Pub. Date: Feb. 26, 2015

(54) MEDIAL TIBIAL PLATEAU REALIGNMENT PROCEDURE FOR MEDIAL OSTEOARTHRITIS OF THE KNEE

(71) Applicant: Peter Stanley Walker, New York, NY (US)

(72) Inventor: Peter Stanley Walker, New York, NY (US)

(73) Assignee: New York University, New York, NY (US)

(21) Appl. No.: 14/457,704

(22) Filed: Aug. 12, 2014

Related U.S. Application Data
(60) Provisional application No. 61/867,797, filed on Aug. 20, 2013.

(51) Int. Cl. A61F 2/38; A61B 17/17

(52) U.S. Cl. CPC A61F 2/389; A61B 17/1764
USPC 623/20.32

(57) ABSTRACT

A method and apparatus for correcting varus deformity comprises cutting a horizontal slot in the upper medial portion of the tibia. The slot is opened by the application of external force and a wedge is inserted to maintain the opening. The opening of the slot corrects the position and orientation of the deformed medial plateau.

### PRIORITY CLAIM TO PREVIOUS PATENT APPLICATIONS

This non-provisional U.S. patent application claims the benefit of priority from U.S. provisional patent application Ser. No. 61/867,797, filed 20 Aug. 2013, the contents of which are incorporated by reference in their entirety.

### FIELD OF INVENTION

The present invention generally relates methods and apparatus for surgically realigning the tibial plateau of a knee whose normal function has been degraded by disease or trauma.

### BACKGROUND

Osteoarthritis (OA) on the medial side of the knee is common in older individuals. The main characteristics, which result in pain with function, are wear of the cartilage surfaces, increased subchondral bone density, osteophytes, a degenerated meniscus, and increasing varus deformity. The causes of OA are multifactorial, and can include post-traumatic, biologic, and genetic elements (Brandt 2008). A mechanistic theory can be included among these many factors. The meniscus loses its weight-bearing capacity, increasing the contact pressures on the cartilage surfaces (Arno 2012). The cartilage gradually breaks down and wear occurs on the distal femoral and proximal tibial surfaces. The meniscus becomes extruded further losing its functional capacity. The subchondral bone on the proximal tibia gradually collapses and becomes denser. During this process, there is a gradual increase in the varus deformity which results in more and more load being transmitted to the medial side (Halder 2013). By the time the cartilage has completely worn, leaving bone-to-bone contact, there is usually severe pain and the only practical treatment option is joint replacement.

At the mid-stage of the OA process, there are opportunities for arresting the progression. While treatments such as injections, visco-supplements, bracing, and shoe-wedges can alleviate pain and slow down progression, they do little to treat the underlying problems which are the excessive medial load and the loss of meniscal function. High tibial open-wedge osteotomy, which has been used for many decades, addresses the overall leg alignment and will reduce medial forces (Wright 2005). However it does not address the varus angulation of the medial tibial plateau per se, nor the meniscal problem. In addition, it is a major procedure involving a large exposure, a bone resection from medial to lateral, and a fixation plate. The short-term to medium-term results are satisfactory but results tend to deteriorate with time (Amendola 2010) leading in most cases to a total knee replacement.

It is important to stress the relative indications for an osteotomy vs a total (or partial) knee replacement. For the osteotomy, the cartilage wear is preferably only partial thickness with the expectation that this cartilage will still provide low friction sliding and not deteriorate further, or only at a slow rate. However when the cartilage is completely worn on both the femur and the tibia, a total knee replacement is indicated. If the wear is only confined to the medial compartment, a unicondylar knee replacement may be indicated. Returning to partial cartilage wear, an osteotomy of only the medial tibial plateau can potentially address the problems and disadvantages of the traditional high tibial open wedge osteotomy.

The present invention has been conceived by the inventor, based on the hypothesis that a medial tibial plateau realignment procedure (MTPR) will correct a varus deformity, reducing the overall medial forces and redistributing them more uniformly on the tibial surface. In this scenario, the cartilage has the possibility of regenerating, or at the least, the degeneration process will be arrested.

### BRIEF DESCRIPTION

In non-limiting embodiments, there is presented a method for reducing the varus angle of the medial tibial plateau comprising cutting a generally horizontal slot in a medial portion of a tibia, the generally horizontal cut positioned at a depth below a tibial plateau and parallel to a plane defined by the tibial plateau surface and having a terminus below a medial tibial spine region; cutting a generally vertical cut in a plane perpendicular to the generally horizontal slot extending from the terminus of the generally horizontal slot in the direction of the peak of the medial tibial spine for a length less than the depth; applying an expanding force within the generally horizontal slot sufficient to expand opening of the slot; positioning a spacer in the slot; the spacer dimensioned to maintain the expanded opening; and removing the expanding force.

In this embodiment, the depth of the generally horizontal slot is 6-10 mms below a lowest point on the medial tibial surface and the generally vertical cut ends 10-15 mms below the top of the medial tibial spine. The method may employ a slotted cutting guide to cut the generally horizontal slot and the generally vertical cut. The slotted cutting guide may comprise an outrigger, configured to be attached an anterior portion of the tibia, wherein the connection between the slotted cutting guide and the outrigger provides relative linear and angular adjustments.

In another embodiment, there is disclosed an apparatus comprising a spreading tool that includes a fixation block having a first rigid body with a mounting face defining a substantially planar mounting surface, an outer face parallel to the mounting face, a plurality of mounting holes perpendicularly penetrating the fixation block from the outer face to the mounting face, a first bore hole penetrating upper portion of the fixation block spaced away from and parallel to the plurality of mounting holes; a rod rotatably passing through the first bore hole and extending beyond the fixation body; a plate block having a second rigid body comprising a second bore hole having the same diameter as the first bore hole, the plate block rotatably mounted on the rod adjacent to the fixation block; a plate attached to the plate block such that the plate is held in plane containing the axis of the rod; and a wedge graft having a periphery conformal with a substantially horizontal slot cut in a tibia and a triangular vertical profile, the wedge graft comprises high friction surfaces.

In another non-limiting embodiment there is presented a method for reducing the varus angle of the medial tibial plateau comprising: cutting a generally horizontal slot in a medial portion of a tibia, the generally horizontal cut ending at a point below the tibial spine, the horizontal slot at 6-10 mm below the medial tibial surface; and drilling a bore hole horizontally through a tibia with the entry point at the anterior extreme of the slot below the tibial spine, with the exit point at the posterior extreme of the slot below the tibial spine. mounting a spreading tool to the tibia so that a rod of the spreading tool is positioned in the bore hole and a plate is positioned within the generally horizontal slot; applying torsional force to the plate block thereby causing plate to rotate and expand the generally horizontal slot; inserting spacer into the generally horizontal slot, the spacer maintains the expansion of the generally horizontal slot; removing the spreading tool; inserting a wedge graft into the expanded generally horizontal slot; and removing the spacer from the generally horizontal slot.

### DESCRIPTION OF DRAWINGS

FIG. 1 shows orthogonal views of the proximal tibia aligned to axes in the tibia overall. FIG. 1a is a posterior view of the proximal tibia showing the vertical axis, often called the long axis, and the medial-lateral (ML) axis. Figure lb is a medial view of the proximal tibia showing the vertical axis and the anterior-posterior axis.

FIG. 2 shows an antero-medial view of the proximal tibia with the 3 axes and cutting planes.

FIG. 3 shows an antero-medial view of the proximal tibia with the slotted cutting guide in position.

FIG. 4 shows an antero-medial view of the proximal tibia with the slots cut in the tibia.

FIG. 5 shows an anterior view of the proximal tibia with the spreading tool just entering the anterior portion of the slot.

FIG. 6 shows an anterior view of the proximal tibia with the graft wedge in place.

FIG. 7 shows the graft wedge.

FIG. 8 shows a spreading tool before attachment to a tibia.

FIG. 9 shows a spreading tool mounted to a tibia.

FIG. 10 shows the application of torsional force to the plate block of the spreading tool.

### DETAILED DESCRIPTION OF EMBODIMENTS

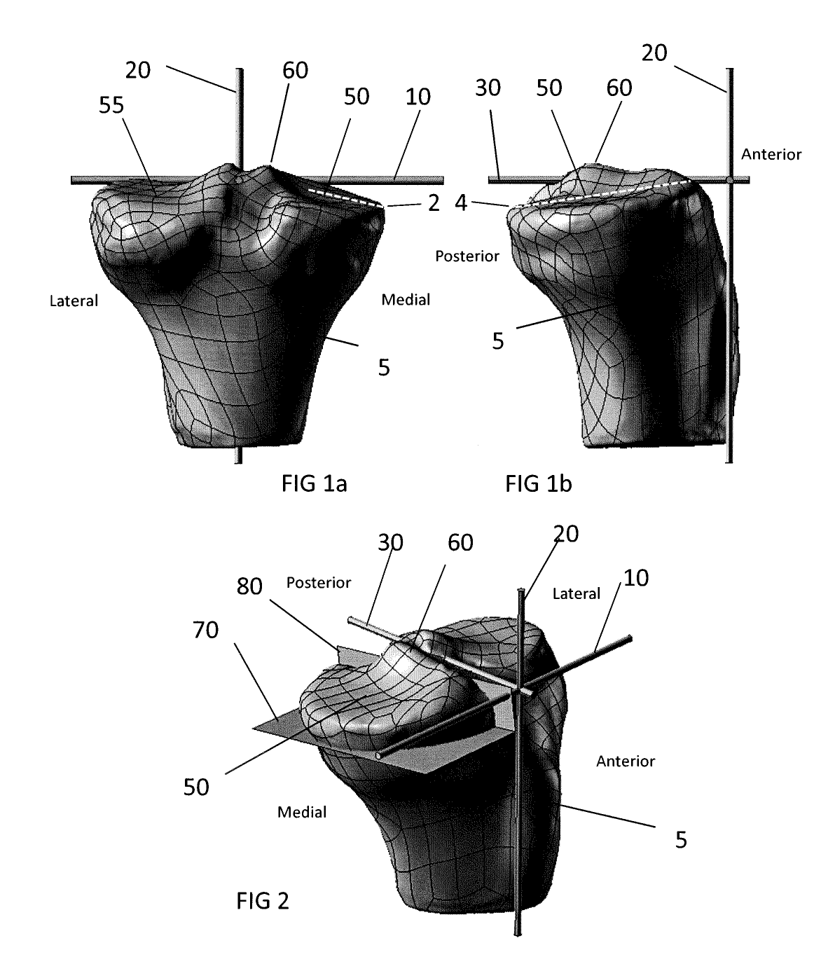

FIG. 1 shows a representative view of a proximal OA tibia 5 determined from an average of 33 OA cases. The tibia 5, is comprised in part, of a medial tibial plateau 50, a lateral tibial plateau 55, and a tibial spine region 60. The medial tibial plateau 50 may, in part, be characterized by a slope 2 in the frontal plane and a slope 4 in the sagittal plane. A vertical axis 20 is defined that passes through the transverse center of the tibia 5 and the transverse center of the ankle (not shown). A medial-lateral axis 10, parallel to the coronal plane, is defined perpendicularly to the vertical axis 20. An anterior-posterior axis 30 is orthogonal to the medial-lateral 10 and vertical 20 axis. The proximal portion of the tibial surface comprised of the medial tibial plateau 50, which typically is slightly dished, and the lateral tibial plateau 55 which, in combination, carry the vertical load. The tibial spine region 60 comprises the bone material between the medial tibial plateau 50 and the lateral tibial plateau 55.

In the posterior view of FIG. 1a, the medial tibial plateau 50 is shown to be sloped medially as indicated by the dashed white line 2. In a normal tibia, the angle of the slope 2 is typically in the range 0-3 degrees relative to the medial-lateral axis 10. This slope is typically called the varus angle. However in OA, the varus angle may be increased by several degrees due to collapse of the subchondral bone. In addition, the partial wear of the cartilage on the surface of the medial tibial plateau 50 may further increase the effective varus angle. When the varus angle is greater than normal, it is called a varus deformity. The effect of deformity is a large increase in the proportion of the vertical load at the knee, carried through the medial tibial plateau 50. This typically causes extra pain and an accelerated rate of wear of the medial cartilage.

It is the purpose of the medial tibial plateau realignment procedure (MTPR), the subject of this invention, to correct the varus deformity, thereby restoring the normal angulation of the medial tibial plateau 50 by making bone adjustments in the area local to the medial tibial plateau 50. This is accomplished by partially slotting the sub-medial portion of the tibia 5 and inserting a spreading tool 112 in the slot. The spreading tool 112 causes the opening of the slot to expand thereby reshaping the damaged tibial platform. The result of the procedure will be a correction of the varus deformity at the knee, and a significant reduction of the medial load, reducing pain and reducing the subsequent rate of cartilage wear.

In the sagittal view of the tibia, FIG. 1b, the tibial plateau is typically sloped from anterior to posterior, called the posterior slope 4. In a normal knee this is usually in the range of 3-7 degrees with reference to anterior-posterior axis 30. In an arthritic knee, this is typically not changed.

FIG. 2 shows two planes, the first being a generally horizontal cutting plane 70, parallel to the plane defined by slopes 2 and 4, and positioned 6-10 mm below the lowest point on the medial tibial surface 50. This depth is selected to be as close as possible to the tibial surface while still preserving the mechanical integrity of the proximal tibia. The depth further provides the possibility that if the MTPR failed in the future, there will still be sufficient bone to successfully perform a total knee replacement. The second is a generally vertical cutting plane 80, parallel to the sagittal plane and perpendicular to horizontal cutting plane 70 is also defined. The generally vertical cutting plane 80 is positioned so that it includes the tip of the tibial spine region 60. As will be described below, the actual cut that will be made, will not extend through the tibial spine 60, but remain at some distance below.

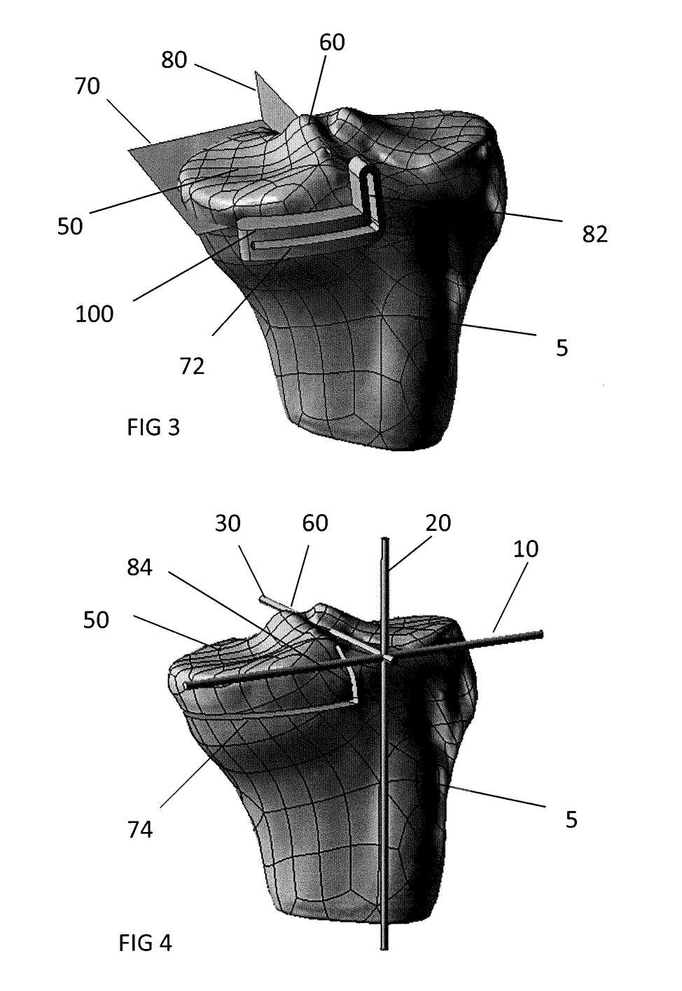

FIG. 3 shows a slotted cutting guide 100 positioned anterior to the medial tibial plateau and aligned with the cutting planes 70 and 80. The method of carrying this out (not shown) is that the slotted cutting guide 100 has an outrigger which is pinned to anterior tibia. The guide 100 is adjustable and pins are used through the horizontal slot 72 to slide over the medial tibial plateau surface 50. When the slotted cutting guide 100 is aligned parallel to the surface of the medial tibial plateau 50, an adjustment is then made to reduce the varus angle. This angle is determined from pre-planning based on an anterior radiograph. Typically the correction across the tibia will be in the range of 2 to 6 degrees. The outrigger will also allow for the slotted cutting guide 100 to be lowered relative to the tibial surface, to a depth between 6-10 mms as noted above. The horizontal slot 72 and the vertical slot 82 will now be aligned for the cuts which are to be made next. Additionally, the top of the vertical slot is adjustable so that the top will be at 10-15 mms below the tip of the tibial spine region 60. The cuts will be made in the bone by passing a 2-3 mms diameter rod-like burring tool through the slots. The horizontal cut could be made with an end-cutting saw, as is common for performing unicondylar or total knee replacement. In that case the vertical cut will need a saw blade of narrow width. The thickness of such saw blades is typically 1.5-2.5 mms.

FIG. 4 shows the slots 74 and 84 cut in the proximal tibia 5. The horizontal cut 74 extends from the medial side to a point below the tip of the tibial spine region 60. The cut also extends from anterior to posterior. The vertical cut 84 extends from the end of the horizontal cut 74 to a point 10-15 mms below the tip of the tibial spine region 60. The purpose of retaining this depth is that the tibial spine region will function as a hinge when the horizontal slot 74 is subsequently opened to correct the varus deformity.

FIG. 5 shows the spreading tool 110 to carry out the opening of the slot 74. The spreading tool 110 consists of a wedge-shaped part 112 which is a similar shape to the bone surface within the horizontal slot 74 and is preferably made of a hard strong material such as a metal. The top and bottom surfaces of the wedge-shaped part 112 are smooth to allow it to slide with only small friction into the slot. A handle 114 is joined to the wedge-shaped part 112, used to direct and impact the entire tool 110 gradually into place in the slot. The leading edges of the wedge-shaped part 112 are slightly beveled to facilitate the introduction of the spreading tool 110. As the spreading tool 110 is gradually introduced, the medial side of the slot 74 will open out, and the bone the tibial spine region 60 will hinge open. The height of this region will be estimated for each case to allow for this opening without too much resistance and without fracturing. In laboratory tests, the 10-15 mms height is the correct range. As the spreading tool 110 is being introduced, if the medial collateral ligament is too tight, it may be necessary to carry some release of the deep fibers of this ligament. This is a common procedure in unicondylar or total knee replacement. However the long fibers of the medial collateral ligament will be preserved. It is noted that the horizontal resection 74 is well above the tibial attachment of this ligament. When the spreading tool 110 has been fully introduced, a flexion-extension test of the knee can be carried out to check for smooth and complete motion. The spreading tool 110 may then be removed.

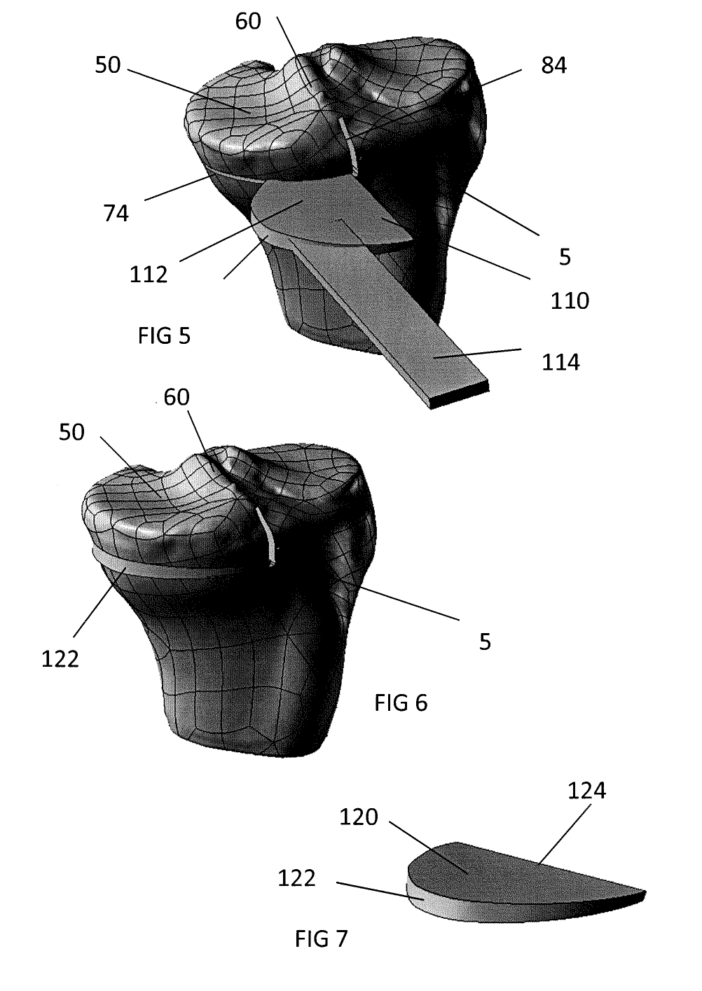

Following removal of the spreading tool 110, the wedge graft 120 may be introduced. FIG. 7 shows an embodiment of the wedge graft 120, prior to implantation. FIG. 6 shows the wedge graft 122 implanted the tibial slot 74. The wedge graft 120 can be made from several materials, but preferably with characteristics of sufficient strength to withstand the vertical compressive force across the medial tibial plateau, and to allow bone osseointegration to form a tight bond with the bone above and below the wedge graft 120. Suitable materials are allograft bone, or a porous metal or ceramic such as with a trabecular structure resembling the cancellous bone of the proximal tibia. Preferably, a small tool (not shown) is made for introduction where the leading edge of the tool is rounded to conform with the shape of the graft, so that when it is tapped into place, there will be no damage. A valgus moment applied across the knee may be applied to facilitate the easy introduction of the wedge graft 120. It will be appreciated that the wedge graft 120 may be made in different sizes and angles.

FIG. 7 shows the shape of the wedge graft 120. The periphery of the wedge graft 120, when viewed from above, is designed to be conforming with the peripheral shape of the proximal tibia, determined from a dimensional study of arthritic tibias from CT or MRI scans. The profile of the wedge graft 120, is triangular. The inside edge of the graft 124 is 2 mms in height equaling the width of the cut made in the bone using the burr or end-cutting saw. The outer edge of the graft 122 is the height necessary to make the angular correction.

The wedge graft 120 is formed with high friction surface finishes that, when brought into contact with the bone surface within the slot, will be sufficient to maintain position of the graft 120. However in some cases it may be elected to place small staples across the slot to maintain the wedge graft 120 in place.

Another non-limiting embodiment is described with the aid of FIGS. 8 through 10. A spreading tool (ST) 210 may be employed to facilitate the procedure.

As previously described, a generally horizontal cutting plane 70, parallel to the plane defined by slopes 2 and 4, and positioned 6-10 mm below the lowest point on the tibial surface 15 is defined. A hole is drilled, from anterior to posterior, through the tibia coaxially with the intersection of the generally horizontal cutting plane 70 and the sagittal plane. A slot is cut in the tibia 5 co-planar with the generally horizontal cutting plane 70 extending from the medial extreme of the tibia to the drill hole. The slotted cutting guide 100 may further a hole drilling guide to properly locate the hole with respect to the slot. The order of drilling the hole and cutting the slot may also be reversed.

The spreading tool 210 comprises four major components as shown in FIG. 8; a fixation block 214, a plate block 232, a plate 230, and a rod 212. The fixation block 214 is a rigid body having a mounting face 260, defining a substantially planer mounting surface, and an outer face 270 parallel to the mounting face 260. A plurality of mounting holes 280 pass perpendicularly from the outer face 270 to the mounting face 260. The upper portion of the fixation block 214 further comprises a first bore hole 290, spaced away from and parallel to the mounting holes 280. The rod 212, which is sized to pass through the hole drilled in the tibia, passes through the first bore hole 290, perpendicularly to the plane of the mounting face 260. The plate block 232 is a rigid body having a second bore hole 300 passing completely through the plate block 232. The rod 212 is also made to pass through the second bore hole 300. The plate 230 is attached to an exterior surface of the plate block 232 and held parallel to the bore hole 300 axis. The plate 230 may comprise a rigid material having a thickness significantly less than that of the slot that is cut in tibia 5. In a non-limiting embodiment, the thickness of plate 230 may be approximately 1 mm. The rod 212 is diametrically dimensioned so that the plate 230 can be rotated around the axis of the rod 212.

Referring to FIG. 9, the spreading tool 210 is shown attached to the tibia 200. The rod 212 is inserted into the hole drilled in the tibia and the fixation block 214 is mounted to the tibia by means of four mounting screws 220 that pass through the mounting holes 280. The plate 230 is inserted into the slot 202 that was cut into the tibia. External clockwise torsional force may be applied to the plate block 232 thereby causing the plate 230 open the slot that was cut in the tibia. The fixation block 214 and the plate block 232 may comprise surfaces adapted to receive levers or wrenches that facilitate the application of torsional force.

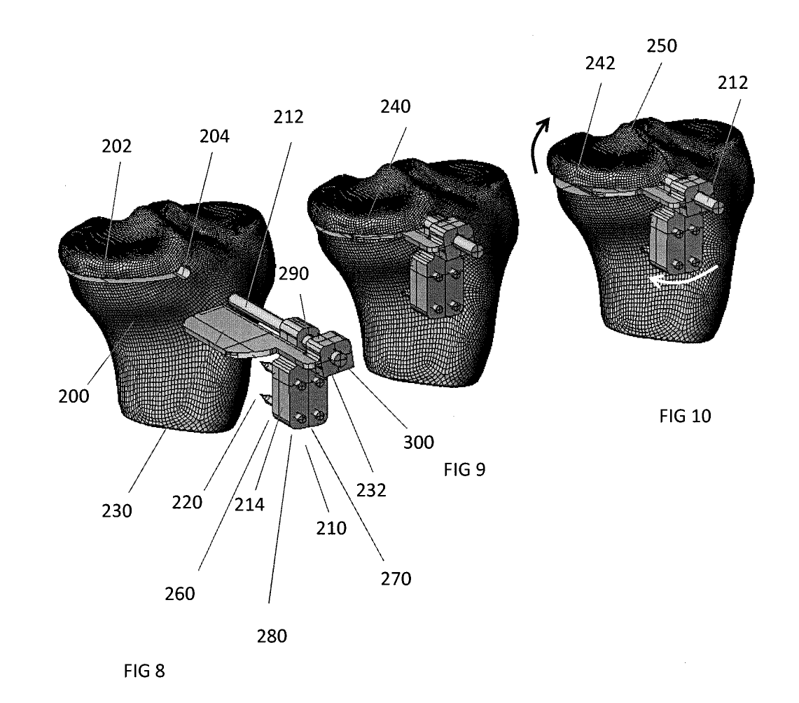

In FIG. 10, the plate 230 and plate block 232 have been rotated clockwise (white arrow) relative to the tibia 200 and the fixation block 214. This has opened up the tibial slot 202 increasing its valgus angle (black arrow). This rotation takes place about the rod 212 and creates a wedge-shaped space at the medial side of the slot 202. A wedge graft 120 made from allograft, autograft, or trabecular metal, may then be introduced into the wedge-shaped space between the plate 230, supporting the underside of the tibial plateau 250, and the tibia 200. The spreading tool 210 may then be removed permitting the slot to close on the wedge graft 230 thereby substantially maintaining the rotated position of the top of the medial tibial plateau 250.

In another non-limiting embodiment a rectangular spacer (not shown) may be initially placed in the opened-up tibial slot 202 to preserve the opening. The spreading tool 210 may then be removed leaving the top of the medial tibial plateau 250 rotated. A wedge graft 120 may then be introduced into the wedge-shaped space between the tibial plateau 250 and the tibia 200. The rectangular spacer may be removed once the wedge graft 120 is introduced.

### Statement Regarding Preferred Embodiments

While the invention has been described with respect to preferred embodiments, those skilled in the art will readily appreciate that various changes and/or modifications can be made to the invention without departing from the spirit or scope of the invention as defined by the appended claims. All documents cited herein are incorporated by reference herein where appropriate for teachings of additional or alternative details, features and/or technical background.

### REFERENCES

Amendola A, Bonasia D E. Results of high tibial osteotomy: review of the literature. International Orthoapedics (SICOT) (2010) 34: 155-160
Arno S, Walker P S, Bell C P, Krasnokutsky S, Samuels J, Abramson S B, Regatte R, Recht M. Relation between cartilage volume and meniscal contact in medial osteoarthritis of the knee. The Knee 2012; 19:896-901
Brandt K D, Dieppe P, Radin E L. Etiopathogenesis of osteoarthritis. Rheum Dis Clin North Am. 2008; 34(3):531-59.
Halder A, Kutzner I, Graichen F, Heinlein B, Beier A, Bergmann G. Influence of limb alignment on mediolateral loading in total knee replacement. J Bone Jt Surg Am, 2012; 94: 1023-9
Wright J M, Crockett H C, Slawski D P, Madsen M W, Windsor R E. High tibial osteotomy. Journal of AAOS, vol 13, no 4, July/August 2005.

What is claimed is:

1. A method for reducing the varus angle of the medial tibial plateau comprising:
cutting a generally horizontal slot in a medial portion of a tibia, said generally horizontal cut positioned at a depth below a tibial plateau and parallel to a plane defined by the tibial plateau surface and having a terminus below a medial tibial spine region;
cutting a generally vertical cut in a plane perpendicular to said generally horizontal slot extending from said terminus of said generally horizontal slot in the direction of the peak of the medial tibial spine for a length less than said depth;
applying an expanding force within said generally horizontal slot sufficient to expand opening of said slot;
positioning a spacer in said slot; said spacer dimensioned to maintain said expanded opening; and
removing said expanding force.

2. The method, in accordance with claim 1, wherein said depth of the generally horizontal slot is 6-10 mms below a lowest point on the medial tibial surface.

3. The method, in accordance with claim 1, wherein said generally vertical cut ends 10-15 mms below the top of the medial tibial spine.

4. The method, in accordance with claim 1, further comprising using a slotted cutting guide to cut said generally horizontal slot and said generally vertical cut.

5. The method, in accordance with claim 4, wherein said slotted cutting guide comprises:
an outrigger, configured to be attached an anterior portion of said tibia, wherein the connection between said slotted cutting guide and said outrigger provides relative linear and angular adjustments.

6. An apparatus comprising:
a spreading tool comprising:
a fixation block having a first rigid body with a mounting face defining a substantially planer mounting surface, an outer face parallel to said mounting face, a plurality of mounting holes perpendicularly penetrating said fixation block from said outer face to said mounting face, a first bore hole penetrating upper portion of said fixation block spaced away from and parallel to said plurality of mounting holes;
a rod rotatably passing through said first bore hole and extending beyond said fixation body;
a plate block having a second rigid body comprising a second bore hole having the same diameter as said first bore hole, said plate block rotatably mounted on said rod adjacent to said fixation block;
a plate attached to said plate block such that said plate is held in plane containing the axis of said rod; and
a wedge graft having a periphery conformal with a substantially horizontal slot cut in a tibia and a triangular vertical profile, said wedge graft comprises high friction surfaces.

7. A method for reducing the varus angle of the medial tibial plateau comprising:
cutting a generally horizontal slot in a medial portion of a tibia, said generally horizontal cut ending at a point below the tibial spine, said horizontal slot at 6-10 mm below the medial tibial surface;
drilling a bore hole horizontally through a tibia with the entry point at the anterior extreme of said slot below the tibial spine, with the exit point at the posterior extreme of said slot below the tibial spine;
mounting a spreading tool to said tibia so that a rod of said spreading tool is positioned in said bore hole and a plate is positioned within said generally horizontal slot;
applying torsional force to said plate block thereby causing plate to rotate and expand said generally horizontal slot;
inserting a wedge graft into said expanded generally horizontal slot; and
removing said spreading tool.
